(19) United States
(12) Patent Application Publication
(10) Pub. No.: US 2022/0161051 A1
(43) Pub. Date: May 26, 2022

(54) LASER HAT WITH ADJUSTABLE FIT

(71) Applicant: LECC TECHNOLOGY CO., LTD., Taoyuan City (TW)

(72) Inventor: HSIN-CHIH TUNG, KINMEN COUNTY (TW)

(21) Appl. No.: 17/203,935

(22) Filed: Mar. 17, 2021

(30) Foreign Application Priority Data
Nov. 23, 2020 (TW) 109215398

(51) Int. Cl. A61N 5/06; H05K 1/18; A42B 1/22; A42B 1/017; A42B 1/019

(52) U.S. Cl. CPC A61N 5/0617; H05K 1/189; H05K 1/181; A61N 2005/067; A61N 5/0619; A42B 1/017; A42B 1/019; A42B 1/22

(57) ABSTRACT

A laser hat with an adjustable fit is provided, which includes a hat body, a plurality of laser modules, a flexible printed circuit board and an adjustment mechanism. The hat body is made of a honeycomb structure. A woven mesh can be used on an inner layer of the hat body. The plurality of laser modules are disposed on the flexible printed circuit board. The flexible printed circuit board is disposed in the hat body and drives the plurality of laser modules, which are used to irradiate a user's head to stimulate hair follicles, and can also be used for laser acupuncture and laser health care treatments for the head. The adjustment mechanism is disposed on one side of the hat body that can be used to adjust a hat fit.

### CROSS-REFERENCE TO RELATED PATENT APPLICATION

This application claims the benefit of priority to Taiwan Patent Application No. 109215398, filed on Nov. 23, 2020. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a laser hat, and more particularly to a laser hat used for laser hair growth treatments, which can effectively mitigate a problem of baldness and hair loss. The laser hat in the present disclosure can also be used for laser acupuncture and laser health care treatments for the head.

### BACKGROUND OF THE DISCLOSURE

Baldness and hair loss have become problems for many people. With the advancement of medical technology, there are more and more ways to treat baldness and hair loss. Clinically, there are two broader categories of treatments, including hair growth treatment and hair transplant surgery. Hair growth treatment includes not only hair growth lotions and oral medicines, but also various kinds of physical therapy and laser hair growth treatment. Laser hair growth has become a widely used treatment in recent years, which can effectively mitigate the problem of baldness and hair loss.

Currently, most laser hair growth devices are designed as a head cover. A plurality of laser modules can be disposed on a hat body, and the plurality of laser modules can emit a laser light to irradiate a user's head to stimulate hair follicles to produce a hair growth effect. However, the conventional laser hair growth device has a hat body of a fixed size, which makes it difficult to be fit on users with different head sizes. Since the user's head size may vary from person to person, a fixed specification may cause an improper fit. However, multiple specifications may also result in usage of too much material, which also causes an increase in cost.

### SUMMARY OF THE DISCLOSURE

In response to the above-referenced technical inadequacies, the present disclosure provides a laser hat with an adjustable fit, which can be adjusted to suit the needs of users with different head sizes.

In one aspect, the present disclosure provides a laser hat, which includes a hat body, a plurality of laser modules, a flexible printed circuit board and an adjustment mechanism. The hat body is a hollow shell and has an inner surface and an outer surface that are respectively arranged on opposite sides of the hat body. The hat body has an opening on one side. The flexible printed circuit board is disposed on the inner surface of the hat body. The plurality of laser modules are disposed on the flexible printed circuit board. The plurality of laser modules are spaced apart from each other and are electrically connected to the flexible printed circuit board. The plurality of laser modules can emit a laser light toward an inside of the hat body. The adjustment mechanism is disposed on the outer surface of the hat body. Two ends of the adjustment mechanism are respectively arranged on opposite sides of the opening so that the adjustment mechanism spans the opening. The adjustment mechanism can adjust an open degree of the opening to adjust a hat fit.

In another aspect, the present disclosure provides a laser hat with an adjustable fit, which includes a hat body, a plurality of laser modules, a flexible printed circuit board, a plurality of first connection members and a plurality of second connection members. The hat body is a hollow shell and has an inner surface and an outer surface that are respectively arranged on opposite sides of the hat body. The plurality of laser modules are disposed on the flexible printed circuit board. The plurality of laser modules are spaced apart from each other and are electrically connected to the flexible printed circuit board. The plurality of laser modules can emit a laser light toward an inside of the hat body. The plurality of first connection members are disposed on the inner surface of the hat body. The plurality of second connection members are disposed on the flexible printed circuit board. The plurality of first connection members can be detachably connected to the plurality of second connection members respectively, so that the flexible printed circuit board can be detachably disposed on the inner surface of the hat body.

Therefore, the laser hat with the adjustable fit provided by the present disclosure can be used to cover a user's head, so that the plurality of laser modules can be used to emit the laser light to irradiate the user's head to stimulate hair follicles that produces a hair growth effect. One side of the hat body has the opening. The adjustment mechanism is disposed on the outer surface of the hat body, and the two ends of the adjustment mechanism are arranged on opposite sides of the opening, so that the adjustment mechanism spans the opening. The adjustment mechanism can be used to adjust an open degree of the opening to provide a proper fit. Therefore, there is no need for a variety of specifications, which may result in too much material being used. In addition, the plurality of laser modules of the laser hat with the adjustable fit provided by the present disclosure can be used to emit the laser light to irradiate the user's head. The laser hat with the adjustable fit provided by the present disclosure can also be used for laser acupuncture, laser health care treatments, and so on for the head.

The hat body in the present disclosure can be made into different sizes. The flexible printed circuit board can be detachably disposed on the inner surface of the hat body. Therefore, various kinds of laser hat with different hat fits can be made by assembling the hat body of different sizes, so as to be suitable for users with different head sizes.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a perspective view of a laser hat with an adjustable fit according to a first embodiment of the present disclosure;

FIG. 2 is a side view of the laser hat with the adjustable fit according to the first embodiment of the present disclosure;

FIG. 3 is a schematic cross-sectional view of the laser hat with the adjustable fit according to the first embodiment of the present disclosure;

FIG. 4 is a detailed view of part IV of FIG. 3;

FIG. 5 is a schematic view of the laser hat with an adjustable fit in a use state according to the first embodiment of the present disclosure;

FIG. 6 is a side view of the laser hat with an adjustable fit in another state according to the first embodiment of the present disclosure;

FIG. 7 is a side view of a laser hat with an adjustable fit according to a second embodiment of the present disclosure;

FIG. 8 is a side view of a laser hat with an adjustable fit according to a third embodiment of the present disclosure;

FIG. 9 is a schematic cross-sectional view of a laser hat with an adjustable fit according to a fourth embodiment of the present disclosure;

FIG. 10 is a detailed view of part X of FIG. 9;

FIG. 11 is a schematic cross-sectional view of a laser hat with an adjustable fit according to a fifth embodiment of the present disclosure; and

FIG. 12 is a schematic cross-sectional view of a laser hat with an adjustable fit according to a sixth embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

### EMBODIMENTS

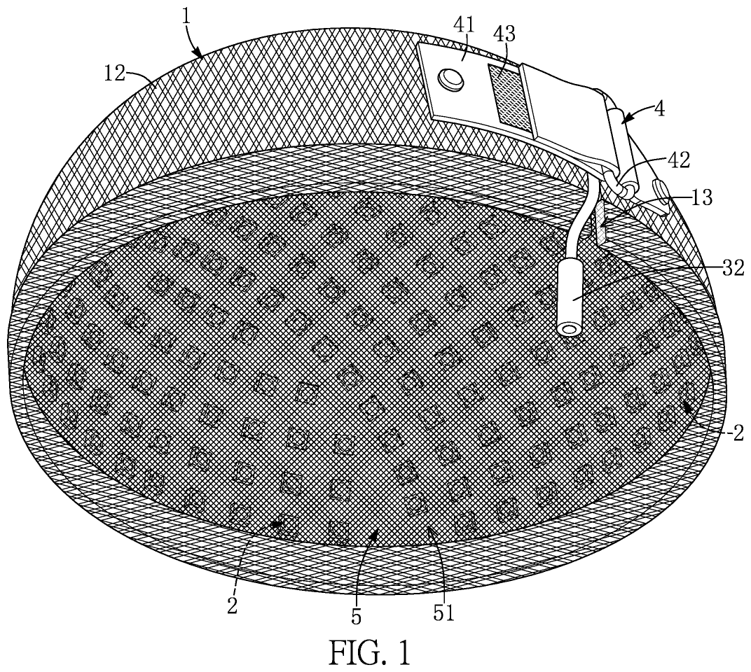

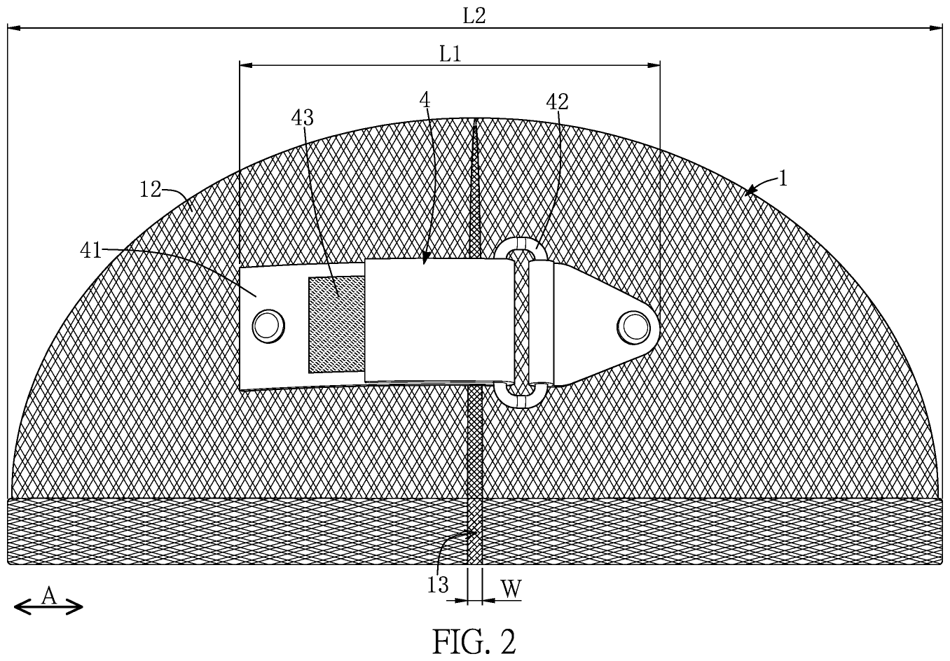

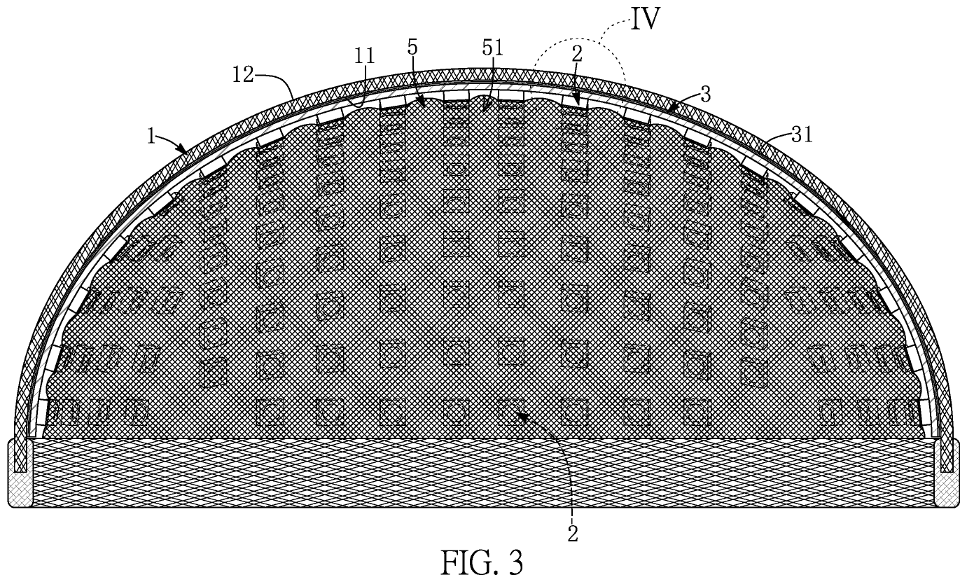

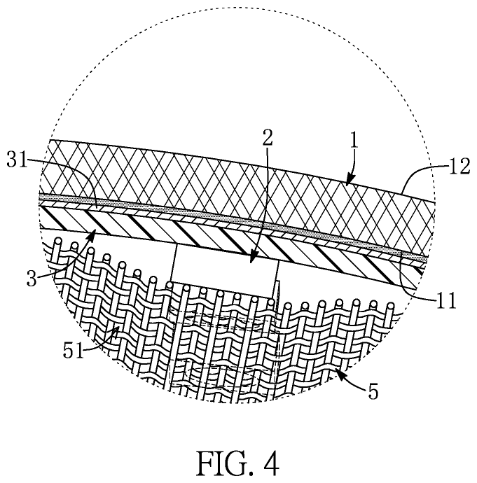

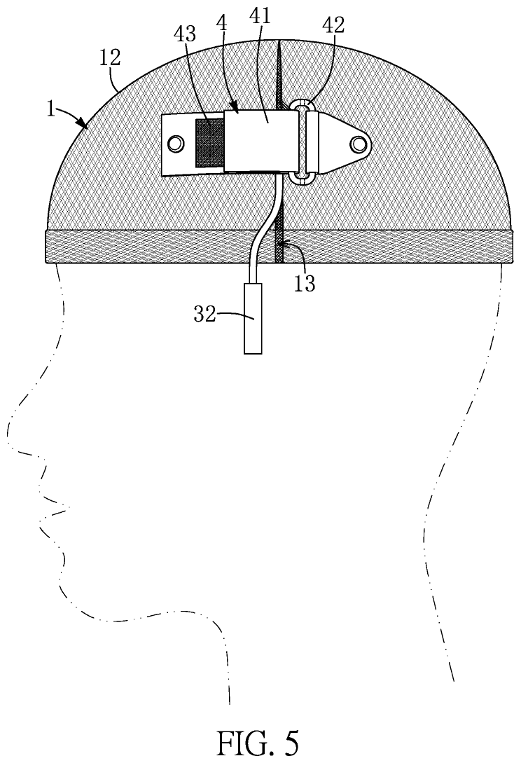

Referring to FIG. 1 to FIG. 5, the present disclosure provides a laser hat with an adjustable fit which can cover a user's head and emit a laser light on the user's head to stimulate hair follicles that causes a hair growth effect. The laser hat with the adjustable fit includes a hat body 1, a plurality of laser modules 2, a flexible printed circuit board 3 and an adjustment mechanism 4.

The hat body 1 is a hollow shell. The hat body 1 can be made of a plastic or the like, and it is not limited herein. Preferably, the hat body 1 can be made of a honeycomb structure, which can be in a lattice shape. A material of the honeycomb structure is a thermoplastic material, which can be a low temperature thermoplastic polymeric material. The low temperature thermoplastic polymeric material has characteristics of easy production, a reduced weight, and a good permeability. In addition, the low temperature thermoplastic polymeric material is strong and durable, easy to manufacture, and can properly protect the flexible printed circuit board 3. The hat body 1 has an inner surface 11 and an outer surface 12. The inner surface 11 and the outer surface 12 are respectively arranged on opposite sides of the hat body 1. The outer surface 12 of the hat body 1 can be left untreated, or can be provided with a variety of colors or patterns to meet customized requirements.

In the present embodiment, the hat body 1 is made of a thermoplastic honeycomb structure, of which a low temperature thermoplastic property is taken advantage of A manufacturing method of the hat body 1 includes the following steps: first, a hat body material is cut into a predetermined shape. Next, the hat body material is heated at a heating temperature of 60° C. to 95° C. for 3 to 12 minutes. After the hat body material is heated and softened, it can be shaped according to a shape of a head, so that the hat body material is formed into the predetermined shape. Then, the heating is stopped and a shaping is carried out for 5 to 25 minutes to produce a hat body 1 with the predetermined shape.

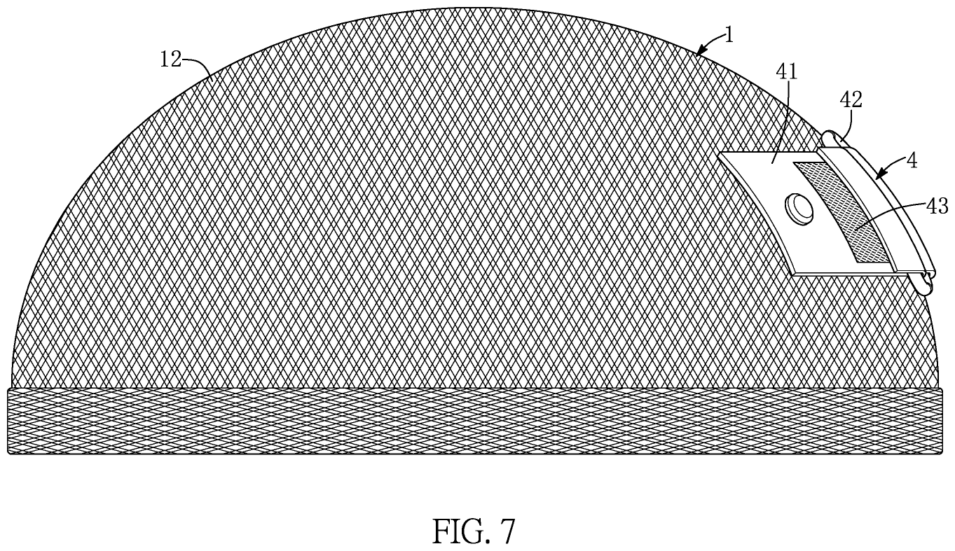

The hat body 1 can be used to cover the user's head, and the inner surface 11 of the hat body 1 faces towards the user's head. The outer surface 12 of the hat body 1 faces away from the user's head, that is, the outer surface 12 of the hat body 1 faces outward. The hat body 1 has an opening 13 on one side, and the opening 13 can be arranged on a left side, a right side, an anterior side, a rear side, or any position of the hat body. In the present embodiment, the opening 13 is arranged on a left side of the hat body 1. In another embodiment, the opening 13 is arranged on a rear side of the hat body 1 (as shown in FIG. 7). The opening 13 passes through the inner surface 11 and the outer surface 12, and the opening 13 can extend from a bottom end to a top end of the hat body 1. An open degree of the opening 13 can be adjusted flexibly so that the hat body 1 can be enlarged or reduced according to the user's head size.

The flexible printed circuit board 3 can be cut into a predetermined shape and appropriately bent and bonded to form a solid shape that corresponds to the inner surface 11 of the hat body 1, so as to be disposed on the inner surface 11 of the hat body 1. The plurality of laser modules 2 are preferably surface-mount device (SMD) type laser modules, thereby reducing a weight and a protrusion of particles, and it is not limited herein. The plurality of laser modules 2, for example, can also be a vertical-cavity surface-emitting laser (VCSEL) module, from which a laser light is emitted perpendicularly to one side. The plurality of laser modules 2 are disposed on the flexible printed circuit board 3. The plurality of laser modules 2 can be disposed on a side of the flexible printed circuit board 3 away from the hat body 1. The plurality of laser modules 2 are spaced apart from each other and are electrically connected to the flexible circuit board 3. The plurality of laser modules 2 are distributed at intervals across the flexible printed circuit board 3, so that the plurality of laser modules 2 are distributed across the laser hat to achieve a wider irradiation area. The flexible printed circuit board 3 can include a driving circuit 31 for driving the plurality of laser modules 2, so that the plurality of laser modules 2 can emit the laser light. The plurality of laser modules 2 can emit the laser light toward the inside of the hat body 1 to irradiate the user's head.

The flexible printed circuit board 3 is disposed on the inner surface 11 of the hat body 1. The flexible printed circuit board 3 can be disposed on the inner surface 11 of the hat body 1 through adhesive bonding, magnetic attraction, a buckle, a hook and loop fastener, etc., but it is not limited herein. The flexible printed circuit board 3 has a flexibility and is near the inner surface 11 of the hat body 1. Since the flexible printed circuit board 3 has the flexibility, it can be properly attached to the inner surface 11 of the hat body 11 along a curvature of the inner surface 11 of the hat body 1. The flexible printed circuit board 3 can also be electrically connected to an electrical connector 32 to facilitate transmissions of an electrical energy and a signal. The electrical connector 32 can be further electrically connected to a conventional controller (omitted from the figures) to control a switching, a timing and a counting operation. The controller can use a battery or a utility power to supply a required electrical energy. Since the controller is conventional technology and is not the focus of the present disclosure, it will not be described in further detail herein.

Preferably, some or all of the plurality of laser modules 2 can be a multi-wavelength laser, which can be used to emit a laser light with various wavelengths. The plurality of laser modules 2 can emit a laser light with two, three, four or five wavelengths. The laser module 2 of the present embodiment is a multi-wavelength laser which can be used to emit a laser light with various wavelengths. A laser light with different wavelengths has different penetration depths so that the laser light can irradiate hair follicles of various depths to achieve a better hair growth effect.

In the present embodiment, the laser hat with adjustable fit further includes a woven mesh 5. The woven mesh 5 is flexible and can cover the plurality of laser modules 2. The woven mesh 5 can be fixed in the hat body 1. The woven mesh 5 is a mesh-like body, and the woven mesh 5 has a plurality of meshes 51. The laser light emitted by the plurality of laser modules 2 can be emitted outward through the corresponding meshes 51. The woven mesh 5 can be used to protect the plurality of laser modules 2 and to prevent the plurality of laser modules 2 from directly contacting a human body, which provides better comfort and safety during use. The woven mesh 5 disposed on an inner layer of the laser hat of the present disclosure can provide weight reduction without affecting the emitted light, which alleviates a problem of optical power loss caused by using a plastic or a silicone in the past.

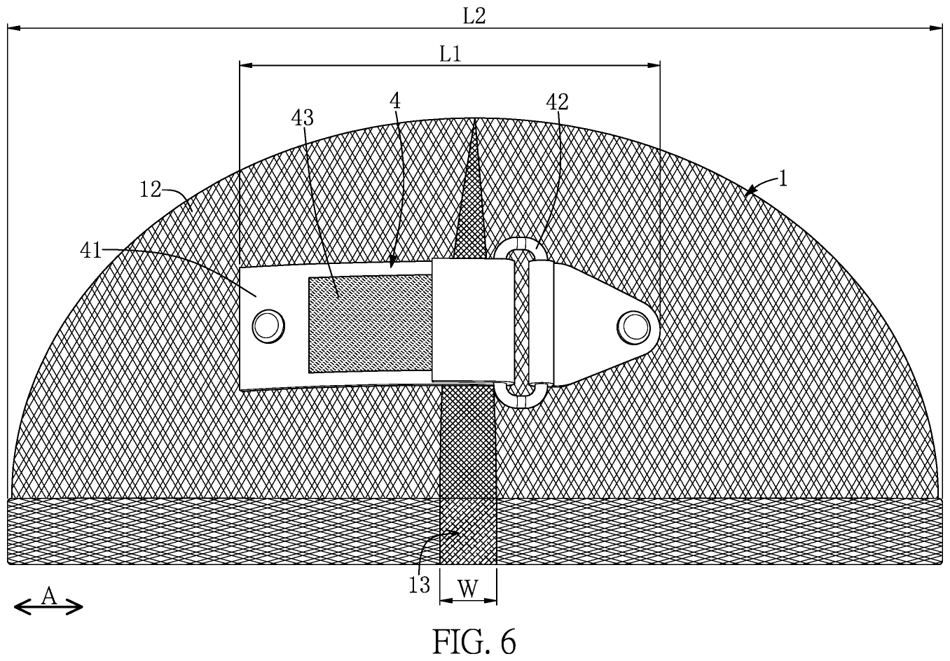

The adjustment mechanism 4 is disposed on the outer surface 12 of the hat body 1. Two ends of the adjustment mechanism 4 are arranged on opposite sides of the opening 13 so that the adjustment mechanism 4 spans the opening 13. In addition, the adjustment mechanism 4 can be arranged between the bottom end and the top end of the hat body 1. The adjustment mechanism 4 can be used to adjust the open degree of the opening 13 by tightening and loosening the opening 13, so as to provide a different degree of tightness of the hat body 1, and can be suitable for users of different head sizes. The hat body 1 can be molded with a larger opening 13 by default, so that the opening 13 of the hat body 1 has a larger opening width W in the normal state (as shown in FIG. 6). The adjustment mechanism 4 can be used to pull the opening 13 to reduce the opening width W, so that the opening width W is reduced, or loosens the opening 13 to enlarge the opening width W, so that the opening width W is enlarged. In the present embodiment, the adjustment mechanism 4 can apply a force in an adjustment direction A to the hat body 1 to pull the opening 13 to reduce the open degree, or to loosen the opening 13 to enlarge the open degree. The adjustment direction A can be parallel to or inclined to a horizontal direction. A structure and an operation of the adjustment mechanism 4 are not limited herein. The adjustment mechanism 4 can be a hook and loop fastener, a magnet or a structure similar to a belt. Preferably, a length L1 of the adjustment mechanism 4 is less than a length L2 of the hat body 1 in a front and rear direction (a longitudinal direction). The adjustment mechanism 4 only needs to span the opening 13 and can be used to tighten and loosen the opening 13 without being too long.

In the present embodiment, the adjustment mechanism 4 can include a belt body 41 and a ring body 42. One end of the belt body 41 is fixed to the outer surface 12 of the hat body 1 and is arranged at one side of the opening 13. The ring body 42 is fixed to the outer surface 12 of the hat body 1 and is arranged at another side of the opening 13. A free end is formed at another end of the belt body 41. The another end of the belt body 41 passes through a predetermined length of the ring body 42 and is reversely folded. The another end of the belt body 41 is adhered to an outer side of the belt body 41 through a hook and loop fastener 43, so that the adjustment mechanism 4 can be used to control the opening degree of the opening 13 to adjust the hat fit of the laser hat according to requirements. The hook and loop fastener 43 can be replaced by a magnet or a buckle. The laser hat with the adjustable fit of the present disclosure can cover the user's head, so that the plurality of laser modules 2 can be used to emit the laser light to irradiate the user's head.

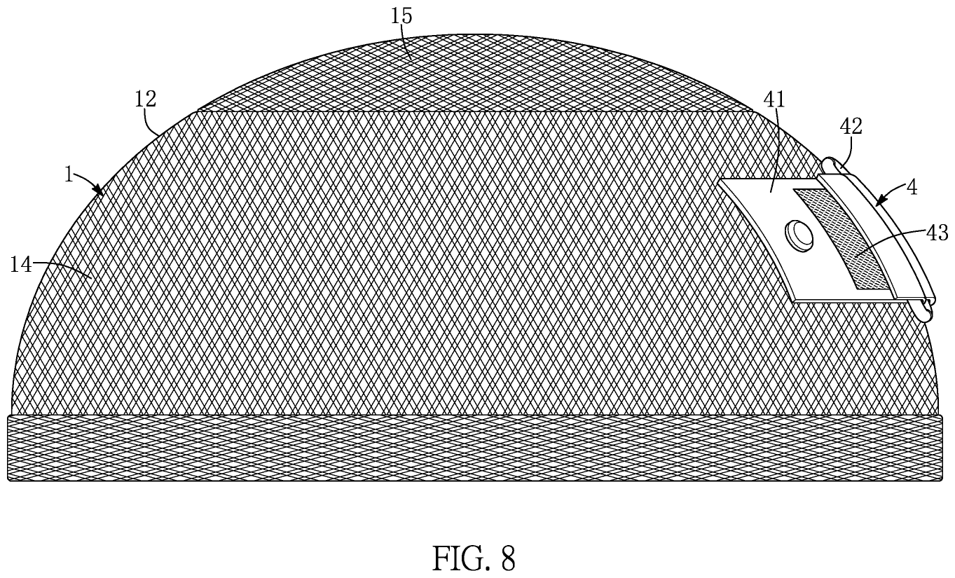

In addition, referring to FIG. 8, the hat body 1 in the present embodiment can have a modular design. The hat body 1 can include a first part 14 and a second part 15. The first part 14 and the second part 15 can be formed individually, and then be assembled by stitching, adhesive bonding, etc. The first part 14 and the second part 15 can be a lower part and an upper part of the hat body 1 respectively, so that the hat body 1 has a two-piece combination design, but it is not limited herein. The hat body 1 can also have a multi-piece combination design, such as a three-piece or a four-piece combination design.

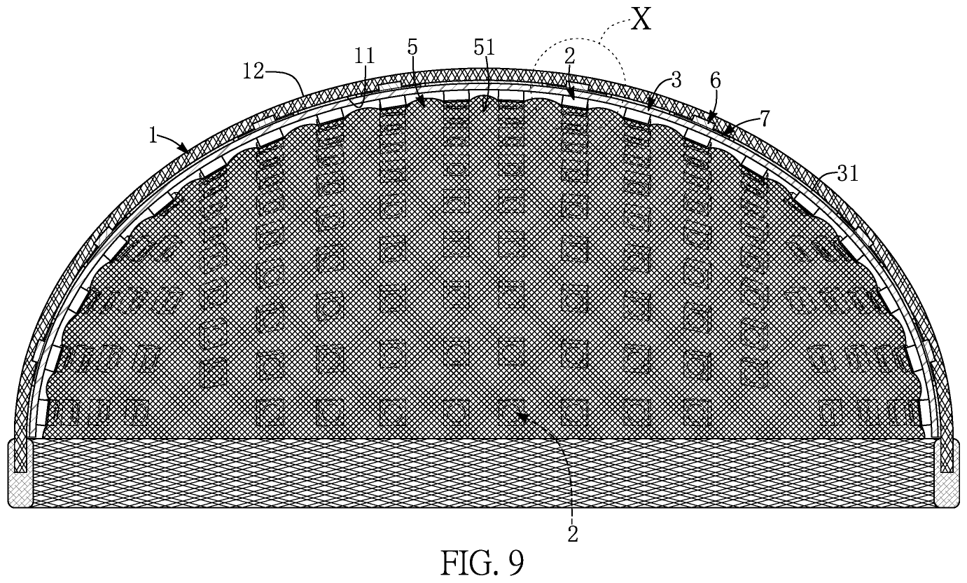

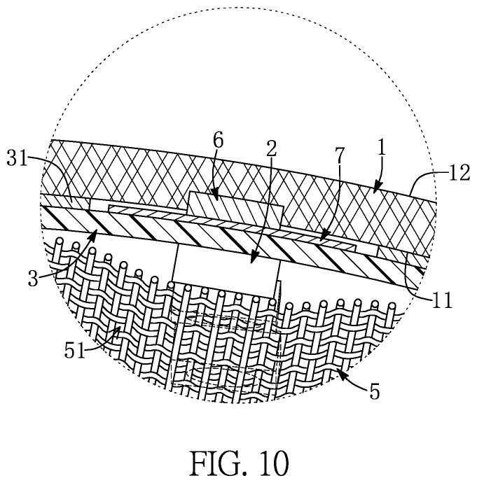

In addition, referring to FIG. 9 and FIG. 10, the flexible circuit board 3 in the present embodiment is detachably disposed on the inner surface 11 of the hat body 1. The flexible circuit board 3 can be disposed on the inner surface 11 of the hat body 1 through magnetic attraction, a buckle, a hook and loop fastener, etc., to facilitate assembly and disassembly. More specifically, the inner surface 11 of the hat body 1 can be provided with a plurality of first connection members 6, and the flexible printed circuit board 3 can be provided with a plurality of second connection members 7. The plurality of second connection members 7 can be disposed on one side of the inner surface 11 of the flexible printed circuit board 3 near the hat body 1. The plurality of second connection members 7 and the plurality of laser modules 2 can be disposed on opposite sides of the flexible printed circuit board 3 respectively, that is, the plurality of second connection members 7 can be disposed on a top surface of the flexible printed circuit board 3, and the plurality of laser modules 2 can be disposed on a bottom surface of the flexible printed circuit board 3. The plurality of first connection members 6 correspond to the plurality of second connection members 7, respectively. The plurality of first connection members 6 are detachably connected to the plurality of second connection members 7 respectively, so that the flexible printed circuit board 3 is detachably disposed on the inner surface 11 of the hat body 1. The hat body 1 in the present embodiment can be made in a variety of sizes, and the flexible printed circuit board 3 can be disposed on the inner surface 11 of the hat body 1 through magnetic attraction, a buckle or a hook and loop fastener. Therefore, it is convenient to change the hat body 1 to different sizes to form various kinds of laser hats with different hat fits, so as to be suitable for users with different head sizes.

In the present embodiment, the flexible printed circuit board 3 is disposed on the inner surface of the hat body 1 through magnetic attraction, that is, the plurality of first connection members 6 and the plurality of second connection members 7 are magnetic attraction components. The first connection member 6 can be a magnet or an iron member, and the second connection member 7 can be an iron member or a magnet. In the present embodiment, the plurality of first connection members 6 are all magnets and the plurality of second connection members 7 are all iron members. The plurality of first connection members 6 and the plurality of second connection members 7 are correspondingly attached to each other so that the flexible printed circuit board 3 can be detachably disposed on the inner surface 11 of the hat body 1.

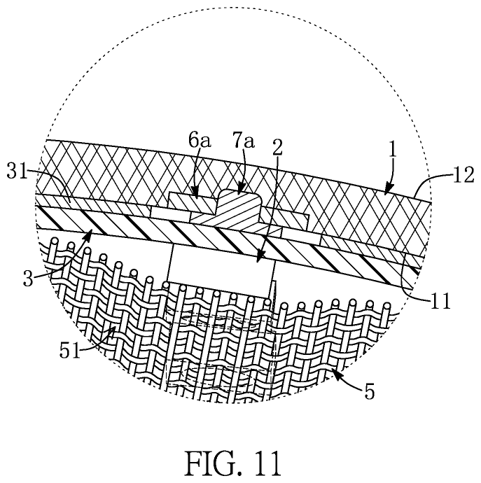

Referring to FIG. 11, the flexible printed circuit board 3 in the present embodiment is disposed on the inner surface 11 of the hat body 1 through a buckle, that is, the plurality of first connection members 6a and the plurality of second connection members 7a are corresponding buckle components. The plurality of first connection members 6a can be all female buckle members and the plurality of second connection members 7a can be all male buckle members. The plurality of first connection members 6a and the plurality of second connection members 7a are correspondingly fastened to each other so that the flexible printed circuit board 3 can be detachably disposed on the inner surface 11 of the hat body 1.

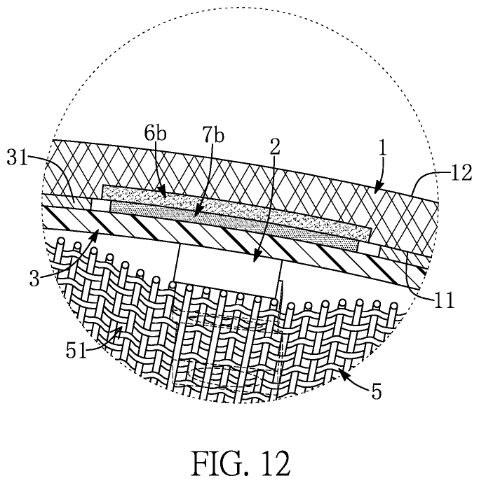

Referring to FIG. 12, the flexible printed circuit board 3 in the present embodiment is disposed on the inner surface 11 of the hat body 1 through a hook and loop fastener, that is, the plurality of first connection members 6b and the plurality of second connection members 7b are corresponding hook and loop fastener components. The plurality of first connection members 6b can be loop structures or hook structures, and the plurality of second connection member 7b can be hook structures or loop structures. The plurality of first connection members 6b and the plurality of second connection members 7b are correspondingly adhered to each other so that the flexible printed circuit board 3 can be detachably disposed on the inner surface 11 of the hat body 1.

### BENEFICIAL EFFECTS OF THE EMBODIMENTS

In conclusion, the laser hat with adjustable fit provided by the present disclosure can be used to cover a user's head, so that the plurality of laser modules can be used to emit the laser light to irradiate the user's head to stimulate hair follicles that produces a hair growth effect. One side of the hat body has the opening. The adjustment mechanism is disposed on the outer surface of the hat body, and the two ends of the adjustment mechanism are arranged on opposite sides of the opening, so that the adjustment mechanism spans the opening. The adjustment mechanism can be used to adjust an open degree of the opening to provide a proper fit. Therefore, there is no need for a variety of specifications, which may result in too much material being used. In addition, the plurality of laser modules of the laser hat with the adjustable fit provided by the present disclosure can be used to emit a laser light to irradiate the user's head. The laser hat with the adjustable fit provided by the present disclosure can also be used for laser acupuncture, laser health care treatments, and so on for the head.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A laser hat with an adjustable fit, comprising:
a hat body, the hat body being a hollow shell, the hat body having an inner surface and an outer surface, the inner surface and the outer surface being respectively arranged on opposite sides of the hat body, and the hat body having an opening on one side;
a plurality of laser modules;
a flexible printed circuit board, the flexible printed circuit board being disposed on the inner surface of the hat body, the plurality of laser modules being disposed on the flexible printed circuit board, the plurality of laser modules being spaced apart from each other, the plurality of laser modules being electrically connected to the flexible printed circuit board, and the plurality of laser modules emitting a laser light toward an inside of the hat body; and
an adjustment mechanism, the adjustment mechanism being disposed on the outer surface of the hat body, two ends of the adjustment mechanism being respectively arranged on opposite sides of the opening so that the adjustment mechanism spans the opening, and the adjustment mechanism adjusting an open degree of the opening to adjust a head fit.

2. The laser hat according to claim 1, further comprising a woven mesh, the woven mesh being flexible and covering the plurality of laser modules, the woven mesh having a plurality of meshes, and the laser light emitted by the plurality of laser modules being emitted outward through the corresponding meshes.

3. The laser hat according to claim 1, wherein the adjustment mechanism includes a belt body and a ring body, one end of the belt body being fixed to the outer surface of the hat body and being arranged on one side of the opening, one end of the ring body being fixed to the outer surface of the hat body and being arranged on another side of the opening, a free end being formed at another end of the belt body, the another end of the belt body passing through a predetermined length of the ring body and being reversely folded, and the another end of the belt body being adhered to an outer side of the belt body with a hook and loop fastener.

4. The laser hat according to claim 1, wherein the hat body is made of a honeycomb structure.

5. The laser hat according to claim 1, wherein the hat body is made of a honeycomb structure and is in a lattice shape.

6. The laser hat according to claim 1, wherein the plurality of laser modules are surface-mount device (SMD) type laser modules.

7. The laser hat according to claim 1, wherein the opening passes through the inner surface and the outer surface, the opening extends from a bottom end to a top end of the hat body, and the adjustment mechanism is arranged between the bottom end and the top end of the hat body.

8. The laser hat according to claim 1, wherein the opening of the hat body has an opening width, and the adjustment mechanism pulls the opening to reduce the opening width so that the opening width is reduced, or loosens the opening to enlarge the opening width so that the opening width is enlarged.

9. The laser hat according to claim 1, wherein the adjustment mechanism applies a force in an adjustment direction to the hat body to pull the opening to reduce the opening width, or loosen the opening to enlarge the opening width, the adjustment direction being parallel to or inclined relative to a horizontal direction.

10. The laser hat according to claim 1, wherein a length of the adjustment mechanism is less than a length of the hat body in a front and rear direction.

11. A laser hat with an adjustable fit, comprising:
a hat body, the hat body being a hollow shell, the hat body having an inner surface and an outer surface, and the inner surface and the outer surface being respectively arranged on opposite sides of the hat body;
a plurality of laser modules;
a flexible printed circuit board, the flexible printed circuit board being disposed on the inner surface of the hat body, the plurality of laser modules being spaced apart from each other, the plurality of laser modules being electrically connected to the flexible printed circuit board, and the plurality of laser modules emitting a laser light toward an inside of the hat body;
a plurality of first connection members, the plurality of first connection members being disposed on the inner surface of the hat body; and
a plurality of second connection members, the plurality of second connection members being disposed on the flexible printed circuit board, the plurality of first connection members being detachably connected to the plurality of second connection members respectively, so that the flexible printed circuit board is detachably disposed on the inner surface of the hat body.

12. The laser hat according to claim 11, further comprising a woven mesh, the woven mesh being flexible and covering the plurality of laser modules, the woven mesh having a plurality of meshes, and the laser light emitted by the plurality of laser modules being emitted outward through the corresponding meshes.

13. The laser hat according to claim 11, wherein the hat body is made of a honeycomb structure.

14. The laser hat according to claim 11, wherein the hat body is made of a honeycomb structure and is in a lattice shape.

15. The laser hat according to claim 11, wherein the plurality of laser modules are surface-mount device (SMD) type laser modules.

16. The laser hat according to claim 11, wherein the plurality of second connection members are arranged on a side of the flexible printed circuit board near the inner surface of the hat body, and the plurality of second connection members and the plurality of laser modules are arranged on opposite sides of the flexible printed circuit board, respectively.

17. The laser hat according to claim 11, wherein the plurality of first connection members and the plurality of second connection members are magnetic attraction components, fastener components or hook and loop fastener components.
